(19) United States
(12) Patent Application Publication
(10) Pub. No.: US 2023/0270907 A1
(43) Pub. Date: Aug. 31, 2023

(54) ROOM REGION FOR AN AIRCRAFT, AIRCRAFT HAVING THE ROOM REGION, AND METHOD FOR DISINFECTING A SURFACE OF A ROOM REGION BY MEANS OF A DISINFECTING DEVICE

(71) Applicant: Diehl Aerospace GmbH, Ueberlingen (DE)

(72) Inventor: Oliver KLETTKE, Nuernberg (DE)

(73) Assignee: Diehl Aerospace GmbH, Ueberlingen (DE)

(21) Appl. No.: 18/020,123

(22) PCT Filed: Sep. 9, 2021
(86) PCT No.: PCT/EP2021/074742

§ 371 Date: Feb. 7, 2023

(30) Foreign Application Priority Data
Sep. 22, 2020 (DE) 10 2020 124 699.4

(51) Int. Cl. A61L 2/26; A61L 2/10

(52) U.S. Cl. CPC A61L 2/26; A61L 2/10; B64D 11/02

(57) ABSTRACT

A room region (1) for an aircraft is provided, which room region comprises a disinfecting device (3) for disinfecting at least one surface (4) of the room region (1), wherein the disinfecting device (3) is designed to disinfect the surface (4) of the room region (1) by means of UVC radiation (5). The disinfecting device (3) comprises a lighting apparatus (6) for generating and emitting the UVC radiation (5). The lighting apparatus (6) is portable, and, in an active state, the lighting apparatus (6) is stationary and arranged.

The invention relates to a spatial region for an aircraft and an aircraft having the spatial region. The invention also relates to a disinfection apparatus for use in a spatial region of an aircraft for disinfecting at least one surface of the spatial region using UVC radiation and to a method for operating a disinfection apparatus, with the disinfection apparatus being designed to disinfect at least one surface of a spatial region of an aircraft.

Washrooms in aircraft are usually cleaned manually by cleaning staff after an aircraft flight. Surfaces with disinfecting cleaning agents can be used to avoid germ formation and kill pathogens. The practice of irradiating surfaces and objects with UVC radiation for disinfection purposes is also already known.

By way of example, document DE 10 2005 027 859 A1 describes a method with apparatuses and arrangements for avoiding the formation of biofilms on the walls of containers, objects, surfaces, etc. in hygiene-relevant dry and wet areas, with short-wavelength UVC radiation from a UVC radiation source being guided through a UVC-conducting matrix and partly being emitted directly by reflection through the surface toward the transporting and surface-wetting medium.

Document WO 2004/108174 A1 discloses a ventilation system for an aircraft cabin and a UVC radiation unit for irradiating the air circulating in the ventilation system with ultraviolet 25 radiation for the purpose of sterilizing the air.

It is an object of the invention to provide an improved solution for disinfecting surfaces in a spatial region in an aircraft. This object is achieved by a spatial region for an aircraft having the features of claim 1, by an aircraft having the spatial region as claimed in claim 10, and by a method for operating a disinfection apparatus having the features of claim 11. Preferred or advantageous embodiments of the invention become apparent from the dependent claims, from the following description, and/or from the appended figures.

A spatial region for an aircraft is proposed. By way of example, the spatial region is designed as a cabin, in particular as a passenger cabin, as an on-board kitchen or galley, or as a washroom or lavatory.

The spatial region comprises a disinfection apparatus. The disinfection apparatus is designed to disinfect at least one surface, preferably a plurality of surfaces, of the spatial region. The surfaces are preferably possible contact areas for passengers and crew members. By way of example, the surfaces can be arranged on a spatial structure of the spatial region, which for example comprises floor, ceiling and walls, and items of equipment and/or furniture. Alternatively or optionally cumulatively, the surfaces may be arranged on sanitary facilities, such as toilets, wash basins or sinks, and on furniture, compartments, seats, etc. The surfaces are optionally arranged in wet and dry areas of the spatial region.

The disinfection apparatus is designed to disinfect the surfaces of the spatial region by means of UVC radiation, preferably by means of short-wavelength UVC radiation, in particular by means of ultraviolet radiation. A wavelength of the UVC radiation is between 280 nanometers and 100 nanometers in particular. The disinfection apparatus for disinfecting the surfaces is preferably arranged within the spatial region, or it is arranged so that it can irradiate and disinfect the surfaces with the UVC radiation. Disinfection using UVC radiation can prevent germ formation and kill pathogens on the surfaces. Thus, infection of people with a disease upon contact with the surfaces can be prevented. In particular, disinfection using UVC radiation can advantageously limit infection events, for example during a prevailing wave of infection and/or a pandemic.

It is advantageous that cleaning staff and/or crew members are not prompted to manually disinfect the surfaces of the spatial region, especially during a flight. Rather, the disinfection apparatus advantageously ensures an independent and preferably regular disinfection of the surfaces. Thus, the cleaning staff and/or the crew members can be unburdened and devote themselves to other important tasks during flight operation.

The disinfection apparatus comprises a luminous device for generating and emitting the UVC radiation. The luminous device preferably comprises at least one fluorescent tube, with the fluorescent tube being designed to generate and emit the UVC radiation. The luminous device, in particular the at least one fluorescent tube, is preferably designed to emit UVC radiation at a wavelength of preferably up to 270 nanometers, in particular up to 260 nanometers, specifically up to 250 nanometers and/or 254 nanometers. The luminous device is optionally designed to irradiate the surfaces with an intensity of at least 50 J/m² (joules per square meter), preferably at least 100 J/m² and/or at most 250 J/m². A radiometric flux and/or current of the UVC radiation is, for example, at least 3.5 watts, preferably at least 4 watts, in particular at least 5 watts and/or 4.2 watts. A duration over which the surfaces are irradiated with the UVC radiation by means of the luminous device for disinfection purposes is at least 20 seconds, preferably at least 30 seconds, in particular at least 40 seconds, and/or at most 60 seconds.

The luminous device is portable, in particular mobile and/or able to be carried along. This applies in particular to a state in which the luminous device is not actively operated, that is to say in which it does not radiate. The luminous device is therefore a piece of portable equipment in its inactive state and can therefore be carried on board an aircraft by a person, if required. In the inactive state, the luminous device can be decoupled from the surface or spatial structure, and it is then a separate, freely movable piece of equipment. The luminous device preferably has manageable dimensions for this purpose, for example dimensions of no more than 400×400×300 millimeters, preferably of no more than 350×350×250 millimeters, in particular of no more than 300×250×150 millimeters and/or of 300×230×100 millimeters. The luminous device preferably has a relatively low weight of no more than 5 kilograms, preferably no more than 3.5 kilograms, in particular no more than 2 kilograms. In particular, on account of the manageable dimensions and the relatively low weight, the luminous device is able to be easily and conveniently transported, in particular carried, into and out of the spatial region.

By way of example, the disinfection apparatus comprises a stowage device in which the luminous device can be stowed away, for example in an inactive state, especially if the luminous device is not in a stand-by state and/or is not generating and emitting any UVC radiation. The stowage device is designed, for example, as a suitcase or trolley, or as a transportable, for example rollable and/or movable, compartment and/or storage compartment of the spatial region. In particular, the luminous device can be accommodated securely and protected from damage in the stowage device. Specifically, the transport of the luminous device into the spatial region and/or out of the latter again can be facilitated by stowing the luminous device away in the stowage device.

In an active state, in particular in a stand-by state and/or in the case of and/or during the generation and emission of the UVC radiation, the luminous device is arranged fixedly and/or stationarily. In this case, fixedly means vis-à-vis the surface to be illuminated and hence be disinfected. In particular, the luminous device is also fixedly arranged vis-à-vis the aircraft cabin or its fittings. Thus, in the active state, the luminous device is affixed to or integrated in the aircraft. Consequently, the same surfaces of the spatial region in particular can be disinfected with each disinfection.

According to the invention, the disinfection apparatus comprises a fastening device. In the active state and by means of the fastening device, the luminous device is fixedly arranged vis-à-vis the surface and on the spatial structure. The luminous device is affixed to the spatial structure by means of the fastening device. The fastening device is designed for fastening the luminous device to the spatial structure of the spatial region, in such a way that it can be detached without damage. The luminous device is detachably connected to the fastening device and/or detachably coupled to the fastening device. By way of example, the luminous device is detachably form-fittingly and/or frictionally connectable to the fastening device. The fastening device consequently acts as an interface between the luminous device and the spatial structure or as an adapter that establishes the connection between the luminous device and the spatial structure. In an inactive state, the luminous device can be decoupled from the fastening device and is then a separate, freely movable piece of equipment, that is to say it is no longer connected to the fastening device and hence the spatial structure. In the decoupled state, the piece of equipment is consequently able to be carried by one person in particular and can be positioned on a further fastening device in the aircraft, or else it can be taken from board.

In a possible implementation of the invention, the luminous device can be detached and/or removed from the fastening device without tools and/or damage. In particular, the luminous device can be easily and quickly connected to the fastening device and detached from the latter again.

The luminous device is preferably removed from the fastening device without being damaged when the said luminous device is inactive and/or in order to stow the said luminous device away in the stowage device. This is advantageous in that the luminous device is connected to the fastening device and fastened to the spatial structure only when required, in particular only when the surfaces are to be disinfected using UVC radiation. In the inactive state, in particular when not in use, the luminous device can be detached or removed from the fastening device and/or safely stowed away in the stowage device. This makes it possible to prevent unwanted manipulation and damage to the luminous device caused thereby, in particular. Furthermore, a risk of injury to people, for example passengers in the aircraft and/or the crew members, in the vicinity of the disinfection apparatus and/or in the spatial region as a result of the UVC radiation can be avoided.

The fastening device is designed as a clamping device, for example. The luminous device can preferably be clamped in the clamping device, with the result that it is held and/or carried by the clamping device. As a result of the luminous device being clamped, the latter is securely connected to the clamping device, and the luminous device cannot inadvertently be detached therefrom, for example in the event of manipulation, flight movements and/or turbulence of the aircraft.

Alternatively, the fastening device is designed as an adapter device, for example as an adapter plate. The adapter device and the luminous device preferably comprise corresponding snap-in interfaces for the detachable coupling of the luminous device to the adapter device.

In a preferred structural embodiment of the invention, the fastening device is fastened to the spatial structure of the spatial region, for example to a wall, a door, a floor, a ceiling or to a piece of the fittings and/or furniture in the spatial region. Preferably, the fastening device is not detachable from the spatial structure without being damaged. In particular, the fastening device is permanently fastened to the spatial structure. By way of example, the fastening device is integrally joined to the spatial structure, in particular adhesively bonded or welded to the latter. Alternatively, form-fitting and/or frictional fastening of the fastening device to the spatial structure is possible.

The fastening device preferably has dimensions that are smaller than the dimensions of the luminous device. Consequently, the fastening device is preferably not visible when the luminous device is fastened and/or coupled thereto. By way of example, the fastening device has dimensions of no more than 380×380×10 millimeters, preferably no more than 250×250×8 millimeters, in particular 120×120×3 millimeters and/or 200×200×6 millimeters.

It is particularly preferred within the scope of the invention for the fastening device to be arranged on the spatial structure below an average eye level for humans. By way of example, the fastening device is fastened to the spatial structure below a height of 170 cm, preferably below 160 cm, in particular below 150 cm, as measured from the floor. This has the advantage that direct eye contact with and/or directly gazing at the fluorescent tube of the luminous device can be avoided. As a consequence, it is possible to reduce or prevent injuries to the eyes of people who enter or stay in the spatial region during operation of the luminous device.

In a possible structural realization of the invention, the disinfection apparatus has a rechargeable power source. The power source is, for example, at least one rechargeable battery and/or at least one accumulator, in particular an NiMh battery pack. The disinfection apparatus can consequently be operated independently of an on-board power supply of the aircraft. The rechargeable power source may be integrated in the fastening device and/or in the luminous device. The luminous device preferably has a connector by means of which the power source can be charged. The power source preferably has a capacity which is sufficient for carrying out a plurality of disinfections of the surfaces of the spatial region, for example at least 15 disinfections, preferably at least 20 disinfections, in particular at least 25 disinfections.

In a further possible structural realization of the invention, the disinfection apparatus has a timer. The timer is preferably designed to activate and/or deactivate the disinfection apparatus, in particular the luminous device in the stand-by state, at a specific point in time and/or after a specific period of time. The disinfection apparatus, in particular the luminous device, is preferably able to be switched on for the first time, in particular able to be put into the stand-by state. To this end, the luminous device preferably has a switch-on device, for example an actuating button or actuating switch. Preferably, the timer only starts the generation and emission of the UVC radiation after a certain amount of time after the switch-on device has been actuated, so that the person who has actuated the switch-on device is given enough time to leave the spatial region. Thus, the person can be protected from UVC radiation. By providing the timer, independent and regular disinfection of the surfaces of the spatial region can advantageously be implemented. In particular, following the initial switch-on, no further action by the cleaning staff and/or crew members is necessary for the regular activation and deactivation of the luminous device.

In a preferred embodiment of the invention, the disinfection apparatus has a protection device. The protection device is designed to protect people in the vicinity of the disinfection apparatus, in particular in the spatial region and/or in other spatial regions of the aircraft, from UVC radiation. This is advantageous as UVC radiation can be harmful to a person's skin and eyes and may cause injury.

It is preferable within the scope of the invention for the protection device to comprise at least one screen. The screen is preferably provided on the luminous device for veneering the fluorescent tube. By way of example, the screen can be opened when the luminous device is active, in order to emit the UVC radiation in the direction of the surfaces. In particular, the screen can be closed when the luminous device is inactive, for example when the luminous device has been removed from the fastening device and/or is stowed away in the stowage device, in order to protect the at least one fluorescent tube from damage and/or to protect the people in the vicinity of the luminous device and/or who are handling the luminous device from UVC radiation. In particular, the screen can be opened and/or closed manually or automatically, for example by way of a suitable actuator.

It is also preferable within the scope of the invention for the protection device to comprise a signaling device. The signaling device is preferably designed to emit an optical and/or acoustic warning signal when the luminous device is active, in particular when the luminous device emits the UVC radiation. The signaling device can be arranged on or integrated in the fastening device or the luminous device. The signaling device which emits the optical warning signal can be, for example, at least one light-emitting diode or any other light source. In particular, the optical warning signal is a continuous or flashing warning light, for example in the signal color red.

The signaling device which emits the acoustic warning signal can be, for example, a loudspeaker. In particular, the acoustic warning signal is a warning tone or a warning noise, for example an easily audible whistle. It is advantageous that people who approach or enter the spatial region are informed that the disinfection apparatus is active and the UVC radiation is being emitted. In particular, people are warned that they are exposing themselves to UVC radiation when they enter the spatial region and/or approach the disinfection apparatus. This can advantageously prevent people from being injured as a result of the UVC radiation.

In a possible realization of the invention, the protection device has a proximity switch. The proximity switch is preferably designed to deactivate the luminous device should a person approach the disinfection apparatus and/or enter the spatial region. The proximity switch preferably comprises a sensor device. The sensor device is preferably designed to detect an approach of the person to the disinfection apparatus as proximity data. To this end, the sensor device is in the form of a motion sensor, for example, which can detect a movement of the person and/or a door in the spatial region. Optionally cumulatively, the proximity switch comprises an actuator which deactivates the luminous device on the basis of the proximity data. It is advantageous that the luminous device is automatically deactivated should the person approach it. This can advantageously prevent injury to the approaching person as a result of the UVC radiation.

Within the scope of the invention, it is possible for the disinfection apparatus to comprise a control device. The control device is designed to control, in particular to activate and deactivate, the timer and/or the protection device, for example the signaling device and/or the proximity switch. Optionally cumulatively, the actuator for opening and closing the screen is controllable by means of the control device.

Further subject matter of the invention relates to an aircraft having the spatial region according to the previous description and/or as claimed in any one of claims 1 to 9.

A disinfection apparatus for use in a spatial region of an aircraft is designed to disinfect the surfaces of the spatial region using UVC radiation. The disinfection apparatus comprises a luminous device, which is designed to generate and emit the UVC radiation. The luminous device is designed to be portable, mobile and/or able to be carried along. Furthermore, in an active state, the luminous device is fixedly and/or stationarily arranged vis-à-vis the surface. The spatial region, in which the disinfection apparatus is used, is preferably designed according to the previous description and/or as claimed in any one of claims 1 to 9.

A method for operating a disinfection apparatus in a spatial region for an aircraft forms further subject matter of the invention. The disinfection apparatus is designed to disinfect at least one surface of the spatial region using UVC radiation. The disinfection apparatus is preferably designed according to the previous description and/or as claimed in claim 11.

The disinfection apparatus comprises a luminous device which generates and emits the UVC radiation after the disinfection apparatus has been put into operation, for example been activated. The disinfection apparatus comprises a fastening device which is designed in particular to fasten the luminous device to a spatial structure of the spatial region so that the said luminous device can be detached without damage. Within the scope of the method, the luminous device, in an active state, in particular when it is in a stand-by state and/or generates and emits the UVC radiation, is fixedly and/or stationarily arranged on the fastening device. Within the scope of the method, the luminous device is removed from the fastening device in an inactive state.

By way of example, the luminous device is connected to the fastening device prior to the activation of the disinfection apparatus, in particular for putting the latter into operation, and is consequently arranged in fixed and/or stationary fashion. Preferably, the luminous device and the fastening device are interconnected so as to be detachable without damage.

In particular, following the deactivation of the disinfection apparatus, for example in the case of non-use, the luminous device is removed from the fastening device without being damaged. Optionally, the luminous device is stowed away in a stowage device of the disinfection apparatus after the said luminous device has been removed from the fastening device.

Preferably, within the scope of the method, the UVC radiation is generated and emitted following the activation of the disinfection apparatus, in particular in the active state, with the surface of the spatial region being disinfected by UVC radiation.

Optionally, within the scope of the method, at least one screen, which veneers at least one fluorescent tube of the luminous device, is opened prior to and/or during the putting into operation and/or activation of the luminous device, in particular in order to be able to emit the UVC radiation at the at least one surface by means of the fluorescent tube. Optionally cumulatively, the at least one screen is closed again after the disinfection has been completed and/or when the disinfection apparatus is inactive, in particular in the inactive state of the luminous device.

An optical and/or acoustic signal is preferably emitted within the scope of the method when the disinfection apparatus is active, in particular when the luminous device is in the active state, specifically during the generation and emission of the UVC radiation. In particular, the disinfection apparatus is deactivated within the scope of the method and/or the emission of the UVC radiation is stopped when a person approaches the disinfection apparatus and/or when there is movement in the spatial region.

Further features, effects and advantages of the invention emerge from the following description of a preferred exemplary embodiment of the invention and from the accompanying figures, in which:

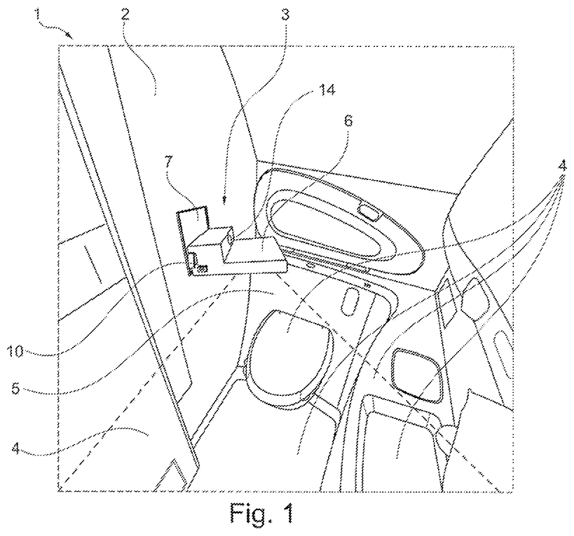

FIG. 1 shows a spatial region with a disinfection apparatus;

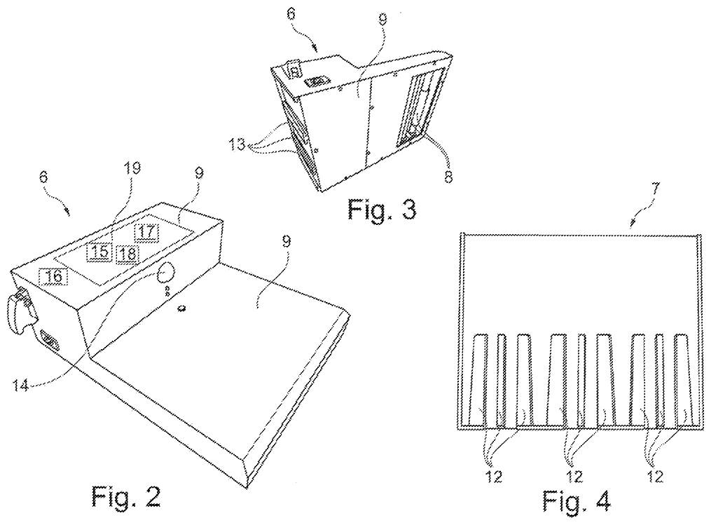

FIG. 2 shows a perspective plan view of a luminous device of the disinfection apparatus from above;

FIG. 3 shows a perspective plan view of the luminous device from below;

FIG. 4 shows a plan view of a fastening device of the disinfection apparatus from the front;

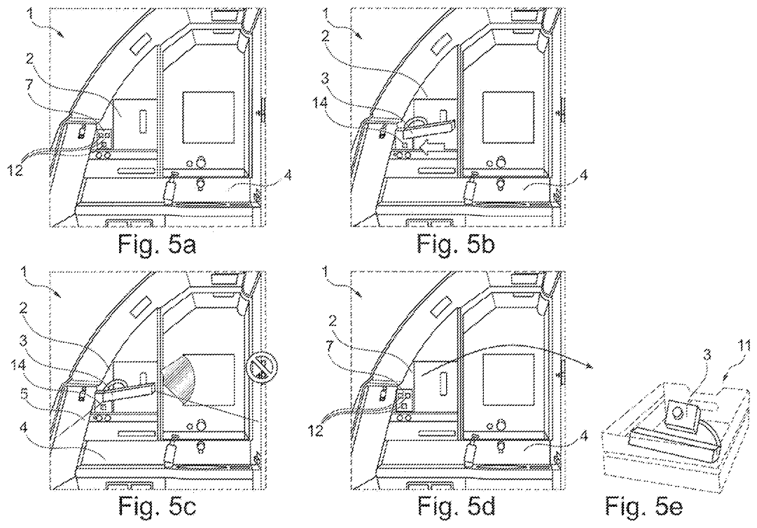

FIGS. 5a-5e show method steps for operating a disinfection apparatus which is designed to disinfect surfaces in a spatial region of an aircraft using UVC radiation.

Corresponding or identical parts are given the same reference signs in the figures.

FIG. 1 shows a spatial region 1 for an aircraft in a perspective view. The spatial region is designed as a washroom and/or as a lavatory. The spatial region 1 comprises a spatial structure 2 comprising side walls, a door, and a floor which at least sectionally delimit the spatial region 1.

The spatial region 1 comprises a disinfection apparatus 3. The disinfection apparatus 3 is designed to implement a disinfection of surfaces 4 of the spatial region 1 using UVC radiation 5. In particular, the surfaces 4 can be sterilized by the disinfection and freed from pathogens, for example viruses and bacteria. The surfaces 4 are wet and dry areas of the spatial region 1. They are sectionally arranged on the spatial structure 2, on a toilet, a 30 washbasin, on furniture and/or on shelves etc., of the spatial region 1.

The disinfection apparatus 3 comprises a luminous device 6. The luminous device 6 is shown in a perspective plan view from above in FIG. 2 and in a perspective plan view from below in FIG. 3.

The luminous device 6 comprises at least one fluorescent tube 8, for example two fluorescent tubes 8. The fluorescent tubes 8 are designed to generate the UVC radiation (FIG. 1). The luminous device 6 can irradiate the surfaces 4 with an intensity of at least 50 J/m², preferably at least 100 J/m² and/or at most 250 J/m² using the UVC radiation 5. The UVC radiation 5 has a wavelength of preferably up to 270 nanometers, in particular up to 260 nanometers, specifically up to 250 nanometers and/or 254 nanometers. A radiometric flux and/or current of the UVC radiation is, for example, at least 3.5 watts, preferably at least 4 watts, in particular at least 5 watts and/or 4.2 watts.

The luminous device 6 comprises a substantially L-shaped receiving housing 9. The fluorescent tubes 8 are accommodated in the receiving housing 9. The receiving housing 9 can comprise at least one movable screen for veneering the fluorescent tubes 8, in particular when the luminous device 6 is inactive. When the luminous device 6 is active, the screen is opened so that the UVC radiation 5 can be emitted. The screen may be designed, for example, as a blind or as a roller shutter.

The luminous device 6 has a relatively low weight of no more than 5 kilograms, preferably no more than 3.5 kilograms, in particular no more than 2 kilograms. The receiving housing 9 has compact and/or manageable dimensions of no more than 400×400×300 millimeters, preferably of no more than 350×350×250 millimeters, in particular of no more than 300×250×150 millimeters and/or of 300×230×100 millimeters. As a result, the receiving housing 9 and hence the luminous device 6 are designed to be mobile and/or portable.

The disinfection apparatus 3 comprises a stowage device 11 (FIG. 5e), in which the luminous device 6 can be stowed away when it is inactive. The stowage device 11 is embodied, for example, as a suitcase, trolley or as a mobile compartment or storage compartment of the spatial region 1, for example.

As shown schematically in FIG. 2, the luminous device 6 comprises a rechargeable power source 15, in particular an accumulator, for example a battery pack. The power source 15 is integrated in the receiving housing 9. A charging socket 10 is arranged on the receiving housing 9, by means of which charging socket the power source 15 can be charged by connecting a cable with a suitable plug. The power source 15 has a capacity by means of which a plurality of disinfections of the surfaces 4, for example at least 15 disinfections, preferably at least 20 disinfections, in particular at least 25 disinfections, can be implemented, preferably with time elapsing in between these.

The disinfection apparatus 3 comprises a fastening device 7, which is shown in FIG. 4 in a plan view from the front. The fastening device 7 is designed as an adapter plate with latching devices 12.

According to FIGS. 1, 5a and 5d, the fastening device 7 is integrally fastened, for example adhesively bonded, to the spatial structure 2, in particular to one of the side walls of the spatial region 1. The fastening device 7 is fastened below a normal eye level of a person, measured for example from a floor of the spatial region 1, at a maximum height of 170 cm, preferably at a maximum height of 160 cm, in particular at a maximum height of 150 cm.

As shown in FIG. 3, the receiving housing 9 of the luminous device 6 comprises counter-latching devices 13 which correspond to the latching devices 12 of the adapter plate. By means of the latching devices 12 and the counter-latching devices 13, the luminous device 6 can be detachably coupled to the fastening device 7, in particular form-fittingly and/or frictionally connected to the fastening device 7 and removed from the latter again without damage. To assemble the disinfection apparatus 3, in particular before it is put into operation and/or activated, the fastening device 7 and the luminous device 6 are detachably coupled to one another, as can be seen in FIG. 5b by virtue of the counter-latching devices 13 being latched in the latching devices 12. In an active state, that is to say when the luminous device 6 is in a stand-by state and/or generates and emits the UVC radiation, the said luminous device is connected to the fastening device 7 and held fixedly and/or stationarily therein vis-à-vis the surface 4.

A switch-on device 14 is arranged on the receiving housing 9 and designed to switch on the disinfection apparatus 3, in particular the luminous device 6, and transition the latter into the stand-by state. The disinfection apparatus 3 can also be switched off again by means of the switch-on device 14. Switching on is implemented manually in particular, for example for the first time after fastening with the fastening device 7. By actuating the switch-on device 14, the disinfection apparatus 3, in particular the luminous device 6, is put into the stand-by state. It is possible for the latter to be switched on, in particular activated, for the first time after it has been coupled to the adapter plate in order to generate and emit the UVC radiation 5.

As shown schematically in FIG. 2, the disinfection apparatus 3 comprises a timer 16, by means of which the luminous device 6 is activatable at a certain point in time and/or after a certain period of time in order to generate and emit the UVC radiation 5. The timer 16 is designed to activate the disinfection apparatus 3 only after a certain period of time after the switch-on device 14 has been actuated. This gives the person who actuates the switch-on device 14 enough time to leave the spatial region 1 before the generation and emission of the UVC radiation 5 starts. In the stand-by state, the luminous device 6 is regularly activatable by the timer 16 for repeated disinfection of the surfaces 4. The disinfection apparatus 3 comprises a control device for controlling the timer 16.

The disinfection apparatus 3 comprises a protection device 19 for protecting people in the vicinity of the disinfection apparatus 3. The protection device 19 comprises the screen, by means of which the fluorescent tube 8 can be veneered in order to protect people from UVC radiation and optionally to protect the fluorescent tube 8 from damage.

The protection device 19 comprises a proximity switch 17. The proximity switch 17 comprises a proximity and/or motion sensor for detecting the approach of the person(s) and/or a movement in the spatial region 1, for example an opening movement of the door. The proximity switch 17 is designed to deactivate the disinfection apparatus 3, in particular the luminous device 6, when one or more persons approach and/or when there is a movement in the spatial region 1 toward the active disinfection apparatus 3. In particular, the proximity switch 17 deactivates the generation and emission of the UVC radiation 5 when the person(s) approach(es) or there is movement in the spatial region 1, in order to prevent the person(s) from being injured by the UVC radiation.

The protection device 19 comprises a signaling device 18. The signaling device 18 is designed to emit an optical signal, for example a warning light and/or flashing light. The signaling device 18 is also designed to emit an acoustic signal, for example a warning tone or warning noise. In particular, the signaling device 18 emits the optical and/or acoustic signal when the disinfection apparatus 3 is active, in particular when the UVC radiation 4 is generated and emitted. Thus, people in the vicinity of the disinfection apparatus 3 who are, for example, in front of, next to or in the spatial region 1 can be warned against entering the spatial region 1, approaching the disinfection apparatus 3 and/or exposing themselves to the UVC radiation 5. The control device is designed to control the protection device 19.

Method steps for operating the disinfection apparatus 3 are shown in FIGS. 5a to 5e. According to FIG. 5a, the fastening device 7 is fastened to the spatial structure 2 and is thus provided and affixed for fastening the luminous device 6 to the spatial structure 2 in such a way that it can be detached without damage.

According to FIG. 5b, the luminous device 6 is detachably coupled to the fastening device 7 and is thereby fixedly and/or stationarily arranged vis-à-vis the surface 4. The switch-on device 14 is actuated in order to switch on the disinfection apparatus 3, in particular the luminous device 6, and/or put the latter into the stand-by state.

According to FIG. 5c, the UVC radiation 5 is generated on account of the switch-on or on account of a switch of the timer 16, and radiated onto the surface 4 for the disinfection of the latter. The surface 4 is irradiated with the UVC radiation 5, for example for at least 20 seconds, preferably at least 30 seconds, in particular at least 40 seconds and/or at most 60 seconds. Then the disinfection is completed. After the certain period of time has elapsed or at the certain point in time, the timer 16 controls one or more renewed disinfections of the surface 4 by emitting the UVC radiation.

The optical and/or acoustic warning signal is emitted during the UVC radiation emission. During the emission, the spatial region 1 is monitored for the approach of a person and/or a movement in the spatial region 1. As soon as the approach and/or movement is detected, the disinfection apparatus 3 is deactivated and the emission of the UVC radiation is stopped or interrupted for a certain amount of time.

After completion of the disinfections, in particular after the deactivation of the disinfection apparatus 3, the luminous device 6 is detached or decoupled from the fastening device 7 according to FIG. 5d, and so the luminous device 6 is now a separate, portable piece of equipment. According to FIG. 5e, the luminous device 6 is stowed away in the stowage device 11. The power source 15 of the disinfection apparatus 3 can be recharged in the stowage device 11 or outside of the latter, with the result that the surfaces 4 of the spatial region 1 can be disinfected again.

### LIST OF REFERENCE SIGNS

1 Spatial region
2 Spatial structure
3 Disinfection apparatus
4 Surfaces
5 UVC radiation
6 Luminous device
7 Fastening device
8 Fluorescent tube
9 Receiving housing
10 Charging socket
11 Stowage device
12 Latching devices
13 Counter-latching devices
14 Switch-on device
15 Power source
16 Timer
17 Proximity switch
18 Signaling device
19 Protection device

1. A spatial region for an aircraft,
the spatial region comprising a disinfection apparatus for disinfecting at least one surface of the spatial region,
the disinfection apparatus being designed to disinfect the surface of the spatial region by means of UVC radiation,
the disinfection apparatus comprising a luminous device for generating and emitting the UVC radiation,
the luminous device being portable, and
the luminous device, in an active state, being fixedly arranged vis-à-vis the surface, the disinfection apparatus comprising a fastening device, the luminous device, in the active state and by means of the fastening device, being fixedly and/or stationarily arranged on a spatial structure of the spatial region and vis-à-vis the surface, and the luminous device being detachable from the fastening device without being damaged,
the luminous device being a portable, separate, freely movable piece of equipment, and
the fastening device being an adapter that establishes the connection between the luminous device and the spatial structure,
the fastening device being permanently fastened to the spatial structure of the spatial region.

2. The spatial region as claimed in claim 1, wherein the disinfection apparatus comprises a stowage device, the luminous device being stowable in the stowage device and, if required, being able to be taken from the stowage device and being able to be coupled to the fastening device.

3. The spatial region as claimed in claim 1, wherein the disinfection apparatus comprises a rechargeable power source which has a capacity for carrying out a plurality of disinfections of the at least one surface.

4. The spatial region as claimed in claim 1, wherein the disinfection apparatus comprises a timer for activation and/or deactivation at a specific point in time and/or after a specific period of time.

5. The spatial region as claimed in claim 1, wherein the disinfection apparatus has a protection device for protecting at least one person from the UVC radiation.

6. The spatial region as claimed in claim 5, wherein the protection device has at least one screen which veneers the at least one fluorescent tube when the luminous device is inactive.

7. The spatial region as claimed in claim 6, wherein the protection device comprises a signaling device for outputting an optical and/or acoustic warning signal when the luminous device is active.

8. The spatial region as claimed in claim 5, wherein the protection device comprises a proximity switch for deactivating the active luminous device should at least one person approach the disinfection apparatus.

9. The spatial region as claimed in claim 1, wherein the spatial region is a cabin, a washroom or a galley for an aircraft.

10. An aircraft having the spatial region as claimed in claim 1.

11. A method for operating a disinfection apparatus of a spatial region as claimed in claim 1 in the spatial region for the aircraft, with the disinfection apparatus being designed to disinfect at least one surface of the spatial region by means of UVC radiation, the disinfection apparatus comprising a luminous device which generates and emits the UVC radiation after the disinfection apparatus has been activated, the disinfection apparatus comprising a fastening device, the luminous device, in an active state, being fixedly arranged on a fastening device and vis-à-vis the surface, and the luminous device being removed from the fastening device when the said luminous device is in an inactive state.
